(19) United States
(12) Patent Application Publication
(10) Pub. No.: US 2004/0241133 A1
(43) Pub. Date: Dec. 2, 2004

(54) Deodorizer agent and deodorizing composition for a mouth cavity, deodorizing food composition and deodorizing composition which contain the deodorizer agent

(76) Inventors: Yasushi Akiyama, Inagawa-cho (JP); Katsuko Nakajima, Nishinamiya City (JP); Satoko Kawai, Fuwa-gun (JP); Shinichi Matsumura, Osaka (JP)

Correspondence Address: FAY KAPLUN & MARCIN, LLP, Suite 702, 150 Broadway, New York, NY, 10038, US

(21) Appl. No.: 10/807,591

(22) Filed: Mar. 24, 2004

(30) Foreign Application Priority Data
May 27, 2003 (JP) 2003-149872

(51) Int. Cl. A61K 35/84; A61K 7/06; C12N 9/02; A61K 35/78; A61K 7/32

(52) U.S. Cl. 424/74; 424/195.15; 424/725; 424/65

(57) ABSTRACT

Described are a deodorizer agent for a mouth cavity with excellent odor eliminating ability, and a composition, a food composition and a deodorizing composition which contain the deodorizer agent. The deodorizer agent includes as an active ingredient a plant derived deodorant component and at least one basidiomycete selected from a group comprising Coriolus hirsutus, Coriolus versicolor, Pynoporus coccineus, Phlebia radiata, Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

### FIELD OF THE INVENTION

The present invention relates to deodorizer agent, deodorizing composition for a mouth cavity, deodorizing food composition and deodorizing composition which contain the deodorizer agent. Purpose of the present invention is to provide the deodorizer agent, deodorizing composition for the mouth cavity and deodorizing food composition which contain the deodorizer agent with highly safety and excellent odor eliminating ability.

### BACKGROUND OF THE INVENTION

In the past, as deodorizer agent for removal foul breath and offensive odor arising from homes and factories etc, deodorizer agent using chemicals such as sodium perborate and hydrogen peroxide, deodorizer agent compounded enzyme such as proteolytic enzyme of papain, and deodorizer agent utilizing deodorizing ability of quinone are provided. However, in these deodorizer agent, deodorizing ability is low, its effect is insufficient, and there is a problem in these deodorizers agent in safety to a human body.

Therefore, deodorizer agent using component included in a food is proposed in consideration of safety to the human body. For example, deodorizer agent that makes hydrophilic solvent extract of mushroom carpophore to be an active component has been shown in an official gazette of Japanese Patent No. 2930323.

Although there is no problem in the deodorizer agent in terms of safety to the human body, but the deodorizing ability is still low and its effect is also inadequacy, hence deodorization ability is insufficient.

The inventors of the present invention eagerly have continued a research in order to solve the above-mentioned problem and obtain the deodorizer agent having sufficient deodorizing ability with highly safety to the human body by containing basidiomycete such as white rot fungi with adding to a plant derived deodorant component, and the present invention has completed.

### SUMMARY OF THE INVENTION

The invention concerning claim 1 relates the deodorizer agent comprising: a plant derived deodorant component and at least one basidiomycete selected from a group comprising of Coriolus hirsutus, Coriolus versicolor, Pynoporus coccineus, Phlebia radiata, Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

The invention concerning claim 2 relates the deodorizer agent described in claim 1, wherein said basidiomycete is a fungus body of the basidiomycete and/or a carpophore of the basidiomycete.

The invention concerning claim 3 relates the deodorizer agent described in claim 1, wherein said plant derived deodorant component is at least one plant extract selected from a group comprising of red alga plant, brown alga plant, gymnosperm and angiosperm.

The invention concerning claim 4 relates the deodorizer agent described in claim 2, wherein said plant derived deodorant component is at least one plant extract selected from a group comprising of red alga plant, brown alga plant, gymnosperm and angiosperm.

The invention concerning claim 5 relates the composition for the mouth cavity, comprising by containing said deodorizer agent described in claim 1.

The invention concerning claim 6 relates the composition for the mouth cavity, comprising by containing said deodorizer agent described in claim 2.

The invention concerning claim 7 relates the composition for the mouth cavity, comprising by containing said deodorizer agent described in claim 3.

The invention concerning claim 8 relates the composition for the mouth cavity, comprising by containing said deodorizer agent described in claim 4.

The invention concerning claim 9 relates the food composition, wherein, comprising by containing said deodorizer agent described in claim 1.

The invention concerning claim 10 relates the food composition, wherein, comprising by containing said deodorizer agent described in claim 2.

The invention concerning claim 11 relates the food composition, wherein, comprising by containing said deodorizer agent described in claim 3.

The invention concerning claim 12 relates the food composition, wherein, comprising by containing said deodorizer agent described in claim 4.

The invention concerning claim 13 relates the deodorizing composition, wherein comprising by containing said deodorizer agent described in claim 1.

The invention concerning claim 14 relates the deodorizing composition, wherein comprising by containing said deodorizer agent described in claim 2.

The invention concerning claim 15 relates the deodorizing composition, wherein comprising by containing said deodorizer agent described in claim 3.

The invention concerning claim 16 relates the deodorizing composition, wherein comprising by containing said deodorizer agent described in claim 4.

### DETAILED DESCRIPTION OF THE INVENTION

Hereinafter, deodorizer agent and deodorizing composition for a mouth cavity, deodorizing food composition and deodorizing composition which contain the deodorizer agent that relate to the present invention are described in detail.

The deodorizer agent of the present invention has a plant derived deodorizing component and the basidiomycete as active ingredients. By containing the plant derived deodorizing component and the basidiomycete as the active ingredients, the deodorizer agent with highly safety and excellent odor eliminating ability can be obtained.

As for the plant derived deodorant component, solvent extract of a red alga plant, a brown alga plant, a gymnosperm and an angiosperm is exemplified. As for these plants, following plant can be exemplified.

As the red alga, a plant belonging to Florideophycidae Gracilariales and Ceramiales Rhodomelaceae can be exemplified.

As the brown alga plant, a plant belonging to Chordariales Nemacystus decipines, Laminariales Laminariaceae, Phaeophyceae Sargassaceae, Laminariales Alaria praelonga can be exemplified.

As the gymnosperm, a plant belonging to Ginkgoales Ginkgoaceae and Coniferae Rachycentridae etc can be exemplified.

As the angiosperm, a plant belonging to Piperales Piperaceae, Fagales Betulaceae, Polygonales Polygonaceae, Ranunculares Magnoliaceae, Ranunculaceae, Ranunculares Lardizabalaceae, Berberidaceae, Lauraceae, Papaverales Papaveraceae, Cruciferae, Sarraceniales Droseraceae, Rosales Hamamelidaceae, Rosaceae, Geraniales Rutaceae, Meliaceae, Parietales Camellia ruticana, Myrtaceae Punicaceae, Myrtaceae, Umbellales Umbelliferae, Elicales Ericaceae, Contortae Oleaceae, Solanales Labiatae, Solanaceae, Scrophulariaceae, Rubiaceae Rubiaceae, Zingiberales Zingiberaceae, Amaranthaceae, Myristicaceae, Araliaceae, Plantaginaceae, Rutaceae and Polygalaceae can be exemplified.

Especially, in the present invention, it is desirable that at least one plant is selected from a group comprising of Salvia officinalis, Scutellaria baicalensis, Eugenia caryophyllata, Rosmarinus officinalis, Origanum majorana, Quercus, Thyinus vulgaris, Eisenia bicyclis, Achyranthes japonica, Myristica fragrans, Acanthopanax henryi, Plantago asiatica, Betula platyphylla, Citrus unshiu, Ecklonia kurome, Polygala tenuifolia and Sargassum thunbergii.

In case of obtaining the plant derived deodorant component from above-mentioned plants, all regios of the plant or each regio of the plant such as leaf, balk, flower, hull, fruit, rhizome and root can be used. Further, depending on the used plant, it is possible to selectively use a regio of the plant containing relatively much deodorization component of the plant.

In order to obtain the plant derived deodorant component from these plants, a well-known method can be used, for example, a method that dried, cut and powdered plant soaks and extracts in extractant, and a method that heats extractant to boiling temperature, reflows and extracts can be exemplified.

The extractant is not especially limited, but water, organic solvent or these mixtures can be exemplified.

As organic solvent, low monohydric alcohol such as methanol, ethanol, propanol etc, polyalcohol, cellosolve, ketone, ester, hydrocarbon, halogenated hydrocarbon and ether can be used.

Further, inorganic acid, organic acid or its aqueous solution, alkalis, saline aqueous solution and buffer solution can be used.

As desirable extractant, water and low monohydric alcohol etc are exemplified, and as more desirable extractant, water and ethanol are exemplified.

In the present invention, it is possible to suitably use mixed solvent of said extractant, and the mixed solvent of water and low monohydric alcohol is preferably used, and the mixed solvent of water and ethanol is used more preferably.

Although, it is usual that extraction liquid obtained from said extraction operation utilizes evaporation solvent as a deodorizer agent, when the solvent is water and ethanol, the solvent is used as the deodorizer without evaporation.

As plant extract, it is possible to use one of said extract independently or mixture of over two kinds extracts (obtained from extraction processing of mixture of the extract or over two kinds plants simultaneously).

In the present invention, as the plant extract, it is desirable to use the plant extract containing phenolic compound. By containing the phenolic compound, higher deodorization ability can be obtained.

As a specific phenolic compound, as long as it has phenolic hydroxyl group, it is not limited especially, for example, catechol, 4-methyl catechol, 5-methyl catechol, resorcinol, chlorogenic acid, caffeine acid, paracumarine, tyrosine, catechin, epicatechin, epigallocatechin, rosmanol, baicalin, carnosol, shikonin etc can be exemplified.

Further, as an active ingredient of the deodorizer agent of the present invention, it can use at least one plant selected from a group comprising of Coriolus hirsutus, Coriolus versicolor, Pynoporus coccineus, Phlebia radiata, Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Tram etes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

All regios of the basidiomycete can be used, and each regio such as bacterial cell and carpophore can be used independently. Further, at least two regios can be used by mixing. In the present invention, liquid culture obtained by cultivating mycelia of said basidiomycete in liquid medium can be used.

By using the liquid culture of the mycelium, compared to use carpophore and bacterial cell, higher deodorization effect can be obtained.

A method for obtaining the liquid culture of the basidiomycete is not limited especially. For example, after inoculum is inoculated in sterilized liquid medium (for example, 1% of glucose, 0.5% of poly peptone, 0.3% of yeast extracts, 0.3% of KH₂PO₄ and 0.1% of MgSO₄H₂O) and the culture is aerobically caltivated at 25° C. about 2 weeks, a method for concentrating culture solution removed bacterial cell and a method that collects the bacterial cell and obtains an objective component by grinding and extraction can be exemplified.

Though the basidiomycete can be used as a dry and comminution material etc, also solvent extract of the basidiomycete can be used. By using the solvent extract of the basidiomycete, compared to use the dry and comminution material of the basidiomycete, it is possible to obtain higher deodorization effect. A method for obtaining the solvent extract of the basidiomycete is not limited especially, and it is possible to exemplify a similar method with the above-mentioned method obtaining a plant derived deodorant component, and a desirable extractant is water or ethanol.

A compounding ratio of the plant derived deodorant component and the basidiomycete is not limited particularly, and 0.001^{˜}10 part by weight, preferably 0.01˜0.2 part by weight of the basidiomycete is mixed to 1 part by weight of the plant derived deodorant component. It is not possible to obtain the excellent deodorization effect, when compounding quantity of the basidiomycete is under 0.0001 part by weight to the plant derived deodorant component of 1 part by weight.

In the present invention, as an active component, the deodorizer contains the plant derived deodorant component and the basidiomycete, it is possible to obtain a deodorizer with excellent deodorization effect by adding dry and comminution material of the basidiomycete and water extract of the basidiomycete to the plant derived deodorant component.

When dry and comminution material of the basidiomycete and water extract of the basidiomycete are added to the plant derived deodorant component, a content ratio is not limited especially, 0.001^{˜}10 part by weight and preferably 0.01˜2 part by weight of the plant derived deodorant component of the basidiomycete and 0.0001^{˜}10 part by weight and preferably 0.001˜2 part by weight of the water extract of the basidiomycete are mixed to 1 part by weight of the plant derived deodorant component.

The deodorizer which relates to the present invention can deodorize offensive odor by applying breath odor, sweat odor and excrement odor of human, pet such as dog, cat and bird, livestock such as cattle, horse and pig.

Further, the deodorizer which relates to the present invention also can deodorize offensive odor which comes from a food such as meat, fish and fermented soybeans which have particular smell, garbage, inside of a refrigerator and industrial waste water.

It is possible to optionally compound a component which is ordinary compounded composition for a mouth cavity composition, when the deodorizer agent of the present invention is utilized to composition for the mouth cavity.

Though compound component is not limited especially, thickening agent, surfactant, bond, abrasive, sweetener, antiseptic, colorant, fragrance and other effective components, etc can be exemplified.

A content of deodorizer effective component in the composition for the mouth cavity is not limited especially, it is 0.001^{˜}5 weight % and preferably 0.01˜2 weight %.

A usage of the composition for the mouth cavity is not limited especially, toothpaste, mouthwash, salve for the mouth cavity, tablet for gargle, troche, chewing tablet, spray for the mouth cavity etc can be exemplified.

When the deodorizer agent which relates to the present invention is utilized by compounding in food composition, it is possible to optionally compound a component in general food.

The compounded component is not limited especially, animal oil, vegetable oil, sweetener, food preservative, fungicide, antioxidants, starch, thickening agent, gelatinizer, starch adhesive, dietary fiber, palatability seasoning, vitamins, kitchen salt, vinegar, soy sauce, spice, enzyme and colorant etc are exemplified.

A content of deodorizer effective component in the food composition is not limited especially, it is 0.001^{˜}20 weight % and desirably 0.01^{˜}2 weight %.

A usage of the food composition is not limited especially, candy, drink, gum, tablet, cookie, cake, frozen dessert, jelly and pasta etc are exemplified.

Except for above-mentioned composition for the mouth cavity and food composition, the deodorizer agent which relates to the present invention can be used as soap, spray, shampoo, rinse, cream, skin lotion for preventing and removing body odor of human body etc which are effective components of cosmetics composition for skin and hair, or can be used as effective component of composition for removing of odor or eliminating odor of shoes, indoor, toilet, pet, car's interior, garbage and cigarette etc.

### EXAMPLES

Hereinafter, the present invention is explained based on the examples, but the invention is not limited to these examples.

(Preparation of Sample 1)

By adding 50% of ethanol of 10 multiples to dry and comminution material of leaf of cha, and it was agitated for 24 hours at room temperature. After extraction liquid was recovered by filtration, extract was recovered by reducing pressure and concentrating.

By adding hexane of 10 multiples to dry and comminution material of leaf stalk of rosemary, it was agitated for 24 hours at room temperature. After extraction liquid was recovered by filtration, extract was recovered by reducing pressure and concentrating.

In adding water to dry and comminution material of carpophore of Coriolus hirsutus, it was agitated for 24 hours at room temperature. After extraction liquid was recovered by filtration, extract was recovered by reducing pressure and concentrating.

In adding water to dry and comminution material of carpophore of Coriolus versicolor, it was agitated for 24 hours at room temperature. After extraction liquid was recovered by filtration, extract was recovered by reducing pressure and concentrating.

In adding water to dry and comminution material of carpophore of Pycnoporus coccineus, it was agitated for 24 hours at room temperature. After extraction liquid was recovered by filtration, extract was recovered by reducing pressure and concentrating.

In adding water to dry and comminution material of carpophore of Phlebia radiata, it was agitated for 24 hours at room temperature. After extraction liquid was recovered by filtration, extract was recovered by reducing pressure and concentrating.

In adding water to dry and comminution material of carpophore of Agaricus bisporus, it was agitated for 24 hours at room temperature. After extraction liquid was recovered by filtration, extract was recovered by reducing pressure and concentrating.

In adding water to dry and comminution material of carpophore of Armillariella mellea, it was agitated for 24 hours at room temperature. After extraction liquid was recovered by filtration, extract was recovered by reducing pressure and concentrating.

In adding water to dry and comminution material of carpophore of Lenzites betulina, it was agitated for 24 hours at room temperature. After extraction liquid was recovered by filtration, extract was recovered by reducing pressure and concentrating.

In adding water to dry and comminution material of carpophore of Daedaleopsis purpurea, it was agitated for 24 hours at room temperature. After extraction liquid was recovered by filtration, extract was recovered by reducing pressure and concentrating.

Coriolus hirsutus was cultured aerobically at 25° C. for two weeks by inoculating in sterilized liquid medium with 0.2% of EBIOS, 0.5% of rice bran, 0.3% of KH₂PO₄, 2% of glucose, 0.5% of poly peptone, 0.3% of yeast extract, 1.3% of NH₄H₂PO₄, 0.5% of MgSO₄·7H₂O. Afterward, objective component was obtained by a method for concentrating culture solution removed bacterial cell or by the grinding extraction collected bacterial cell.

### Test Example 1

In order to confirm a reduction effect of human breath odor, reduction effect of breathe odor under with or without of the basidiomycete was examined on plants shown in table 1. Liquid dissolved 5 mg of plant extract and 1 mg of carpophore of the basidiomycete in 0.05M phosphate buffer 10 ml, which is pH 6.0 as an active control, and it was contained in the mouth cavity and washing out for 30 seconds. After 5 minutes of shut the mouth from washing out, air in the mouth cavity was collected with expiration in 100 ml tedlar bag, and in addition, the air was shifted in 3 litters of Flek-Samplar (smell bag: OMI ODORAIR SERVICE Corp oration).

After odorless air (air passed activated carbon column) was added 2.5 liters, Flek-Samplar was evaluated by 7 functional evaluation panelists immediately. Further, as a control, after contained 10 ml of 0.05M phosphate buffer or 10 ml of 0.05M phosphate buffer only contained 5 mg of plant extract or carpophore of the basidiomycete in the mouth cavity and washing out for 30 seconds, air in the mouth cavity was also similarly adjusted and the function was evaluated.

The air in the mouth cavity was also collected after 5 minutes and 60 minutes after mouth rinsing.

| | |
|---|---|
| Extremely discomfort | 5 |
| Very discomfort | 4 |
| Discomfort | 3 |
| Somewhat discomfort | 2 |
| Not discomfort | 1 |

Also, air that plant extract and the basidiomycete of table 1 was independently used was similarly evaluated.

TABLE 1

| | | | | evaluation of | evaluation of |
|---|---|---|---|---|---|
| | | | evaluation | deodorizing | deodorizing |
| | | | before mouth | after | after |
| | plant extract | basidiomycete | rinsing | 5 minutes | 60 minutes |
| Comparative | tea | — | 3.7 | 2.8 | 3.3 |
| Example 1 | | | | | |
| Comparative | — | Coriolus | 4.0 | 3.6 | 3.8 |
| Example 2 | | hirsutus | | | |
| Embodiment 1 | tea | Coriolus | 3.9 | 1.3 | 1.9 |
| | | hirsutus | | | |
| Comparative | — | Pynoporus | 3.9 | 3.7 | 3.9 |
| Example 3 | | coccineus | | | |
| Embodiment 2 | tea | Pynoporus | 3.9 | 1.5 | 2.4 |
| | | coccineus | | | |
| Comparative | — | Daedalea | 4.0 | 3.9 | 3.9 |
| Example 4 | | dickinsii | | | |
| Embodiment 3 | tea | Daedalea | 4.2 | 1.2 | 1.7 |
| | | dickinsii | | | |
| Comparative | rosemary | — | 4.3 | 2.8 | 3.3 |
| Example 5 | | | | | |
| Embodiment 4 | rosemary | Daedalea | 4.0 | 1.1 | 2.3 |
| | | dickinsii | | | |
| Embodiment 5 | rosemary | Pynoporus | 4.1 | 1.9 | 2.7 |
| | | coccineus | | | |

(Preparation of Sample 2)

By adding 50% of ethanol of 10 multiples to dry and comminution material of leaf stalk of rosemary, and it was agitated for 24 hours at room temperature. After extraction liquid was recovered by filtration, extract was recovered by reducing pressure and concentrating.

In adding water to dry and comminution material of carpophore of Coriolus hirsutus, it was agitated for 24 hours at room temperature. After extraction liquid was recovered by filtration, extract was recovered by reducing pressure and concentrating.

Next, deodorizing composition of embodiment 6 was prepared according to following prescription. Further, one of Coriolus hirsutus or rosemary was added, and other composition were prepared similarly to embodiment 6 and deodorizing composition of comparative example 6 and 7 were prepared.

<deodorizing composition>

| Coriolus hirsutus | 0.001 | |
|---|---|---|
| Rosemary | 0.001 | |
| EDTA | adequate dose | |
| Paraben | adequate dose | |
| Water | rest | |
| Sum | 100.00 | weight % |

### Test Example 2

500 g of garbage were put in a plastic bucket with a lid, which can include 10 liters. After laying lid and 30 minutes had passed, with following to appraisal standard, inside smell was evaluated by 7 function evaluation panelists. Mean value of 7 panelists were calculated.

Next, 10 g of test sample of the embodiment 6, comparative example 6 or comparative example 7 were sprinkled. After 15 minutes and 60 minutes passed from the sample was sprinkled, with following to appraisal standard, inside smell was evaluated by 7 function evaluation panelists. Mean value of 7 panelists were calculated.

<Deodorizing Composition>

1 Not smell

2 Scent

3 Slightly smell

4 Odor

5 Stench

TABLE 2

| | | | evaluation | evaluation | evaluation |
|---|---|---|---|---|---|
| | | | before | after | after |
| | plant extract | basidiomycete | sprinkle | 15 minutes | 60 minutes |
| comparative | rosemary | — | 5.0 | 3.6 | 4.0 |
| example 6 | | | | | |
| comparative | — | Coriolus | 5.0 | 3.8 | 4.8 |
| example 7 | | hirsutus | | | |
| embodiment 6 | rosemary | Coriolus | 5.0 | 2.3 | 2.7 |
| | | hirsutus | | | |

Hereinafter, combination examples for the mouth cavity and food compositions which relate to the present invention are shown.

<Combination example 1; Tooth paste>

| | |
|---|---|
| Aluminium hydroxide: | 43.0 |
| Glycerin: | 20.0 |
| Carboxymethylcellulose sodium: | 2.0 |
| Sodium lauryl sulfate: | 2.0 |
| Fragrance: | 1.0 |
| Saccharin sodium: | 0.1 |
| 30% ethanol extract of Scutellaria: | 0.15 |
| Water extract of Coriolus hirsutus carpophore: | 0.1 |
| N-Lauroyl sarcosine sodium: | 0.2 |
| Water: | rest |
| Sum: | 100.0 weight % |

<Combination example 2; Tooth paste>

| | |
|---|---|
| Dibasic calcium phosphate: | 20.0 |
| Carboxymethylcellulose sodium: | 1.5 |
| Saccharin sodium: | 0.2 |
| Glycerin: | 20.0 |
| Sodium lauryl sulfate: | 1.0 |
| Hexylene glycol: | 5.0 |
| Triclosan: | 0.1 |
| Water extract of Eisenia bicyclis: | 0.1 |
| Water extract of Coriolus versicolor carpophore: | 0.2 |
| Sodium chloride: | 15.0 |
| Fragrance: | 1.0 |
| Water: | rest |
| Sum: | 100.0 weight % |

<Combination example 3; Mouth Washes>

| | |
|---|---|
| Ethanol: | 10.0 |
| Polyoxyethylene hydrogenateded castor oil: | 0.5 |
| Sodium lauryl sulfate: | 0.5 |
| Saccharin sodium: | 0.2 |
| Glycerin: | 10.0 |
| Water extract of rosemary: | 0.1 |
| Water extract of Pycnoporus coccineus | 0.2 |
| carpophore: | |
| Fragrance: | 0.2 |
| Water: | rest |
| Sum: | 100.0 weight % |

<Combination example 4; Troche>

| | |
|---|---|
| Alabic gum: | 6.0 |
| Palatinose: | 30.0 |
| Maltitol: | 20.0 |
| Isomaltose: | 22.0 |
| Gelatine: | 3.0 |
| Fragrance: | 0.2 |
| 50% ethanol extract of Rhubarb: | 0.5 |
| Water extract of Phlebia radiata carpophore: | 0.5 |
| Water: | rest |
| Sum: | 100.0 weight % |

<Combination example 5; Chewing gum>

| | |
|---|---|
| Gum base: | 100 part by weight |
| Citric acid: | 1 part by weight |
| Fragrance: | 1 part by weight |
| Water extract of white birch: | 1 part by weight |
| 50% ethanol extract of Agaricus bisporus | 1 part by weight |
| carpophore: | |

<Combination example 6; Juice>

| | |
|---|---|
| Concentrated fruit juice: | 5.0 part by weight |
| Fructose, glucose and liquid sugar: | 10.0 part by weight |
| L-ascorbic acid: | 0.2 part by weight |
| Water: | 85.0 part by weight |
| Fragrance: | 0.1 part by weight |
| 50% ethanol extract of Armillariella mellea | 0.5 part by weight |
| carpophore: | |
| Water extract of clove: | 1.0 part by weight |

<Combination example 7; Candy>

| | |
|---|---|
| Sugar: | 120.0 part by weight |
| Starch syrup: | 100.0 part by weight |
| Citric acid: | 4.0 part by weight |
| Fragrance: | 0.4 part by weight |
| 50% ethanol extract of cassia: | 1.0 part by weight |
| 50% ethanol extract of Lentinus edodes | 1.0 part by weight |
| carpophore: | |

<Combination example 8; Biscuit>

| | |
|---|---|
| Hard flour: | 100.0 part by weight |
| Weak flour: | 100.0 part by weight |
| White superior soft sugar: | 30.0 part by weight |
| Powder sugar: | 40.0 part by weight |
| Shortening: | 100.0 part by weight |
| Baking soda: | 0.6 part by weight |
| Whole milk powder: | 4.0 part by weight |
| Water: | 20.0 part by weight |
| 50% ethanol extract of thyme: | 1.0 part by weight |
| 50% ethanol extract of Lenzites betulina | 1.0 part by weight |
| carpophore: | |

As explained above, the deodorizer agent which relates to the present invention has excellent deodorization ability than using a plant derived deodorant component independently.

The composition for the mouth cavity, food composition and deodorizing composition containing the deodorizer agent which relates to the present invention has excellent deodorizing ability and higher safety.

1. A deodorizer agent comprising:
a plant derived deodorant component and
at least one basidiomycete selected from a group comprising of Coriolus hirsutus, Coriolus versicolor, Pynoporus coccineus, Phlebia radiata, Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

2. The deodorizer agent described in claim 1, wherein said basidiomycete is at least one of a fungus body of the basidiomycete and a carpophore of the basidiomycete.

3. The deodorizer agent described in claim 1, wherein said plant derived deodorant component is at least one plant extract selected from a group comprising of red alga plant, brown alga plant, gymnosperm and angiosperm.

4. The deodorizer agent described in claim 2, wherein said plant derived deodorant component is at least one plant extract selected from a group comprising of red alga plant, brown alga plant, gymnosperm and angiosperm.

5. A composition for a mouth cavity, comprising:
a deodorizer agent including a plant derived deodorant component and at least one basidiomycete, the at least one basidiomycete being selected from a group comprising Coriolus hirsutus, Coriolus versicolor, Pynoporus coccineus, Phlebia radiata. Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

6. The composition for the mouth cavity described in claim 5, wherein said basidiomycete is at least one of a fungus body of the basidiomycete and a carpophore of the basidiomycete.

7. The composition for the mouth cavity described in claim 6, wherein said plant derived deodorant component is at least one plant extract selected from a group comprising red alga, brown alga, gymnosperm and angiosperm.

8. The composition for the mouth cavity described in claim 7, wherein said plant derived deodorant component is at least one plant extract selected from a group comprising red alga, brown alga gymnosperm and angiosperm.

9. A food composition, comprising: a deodorizer agent including a plant derived deodorant component and at least one basidiomycete, the at least one basidiomycete being selected from a group comprising Coriolus hirsutus, Coriolus versicolor, Pynoporus coccineus, Phlebia radiata, Agaricus bisporus, Armillariella mellea, Lentinus edodes, Lenzites betulina, Daedalea dickinsii. Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

10. The food composition described in claim 9, wherein said basidiomycete is at least one of a fungus body of the basidiomycete and a carpophore of the basidiomycete.

11. The food composition described in claim 10, wherein said plant derived deodorant component is at least one plant extract selected from a group comprising red alga, brown alia, gymnosperm and angiosperm.

12. The food composition described in claim 11, wherein said plant derived deodorant component is at least one plant extract selected from a group comprising red alga, brown alga, gymnosperm and angiosperm.

13. A deodorizing composition, comprising: a deodorizer agent including a plant derived deodorant component and at least one basidiomycete, the at least one basidiomycete being selected from a group comprising Coriolus hirsutus, Coriolus versicolor, Pynoporus coccineus, Phlebia radiata, Agaricus bisporus, Armillariella mellea, Lentinus edodes, Lenzites betulina, Daedalea dickinsii, Trametes acupunctata, Trametes atypa, Trametes biokoensis, Trametes blumei, Trametes campestris, Trametes carbonaria, Trametes cingulata, Trametes colliculosa, Trametes conchifera, Trametes cristata, Trametes devexa, Trametes drummondii, Trametes foedata, Trametes friesii, Trametes gibbosa, Trametes heteromorpha, Trametes hirsuta, Trametes hispida, Trametes hispidans, Trametes lactinea, Trametes mariarina, Trametes menziesii, Trametes meyenii, Trametes mollis, Trametes muelleri, Trametes obstinata, Trametes occidentalis, Trametes ochracea, Trametes orientalis, Trametes paleacea, Trametes persoonii, Trametes pinsita, Trametes proteiformis, Trametes rigida, Trametes rubescens, Trametes scabrosa, Trametes scobinacea, Trametes serpens, Trametes suaveolens, Trametes subsinuosa, Trametes trogii, Trametes ungulata, Trametes varians, Trametes velutina, Trametes versicolor and Trametes zonata.

14. The deodorizing composition described in claim 13, wherein said basidiomycete is at least one of a fungus body of the basidiomycete and a carpophore of the basidiomycete.

15. The deodorizing composition described in claim 14, wherein said plant derived deodorant component is at least one plant extract selected from a group comprising red alga, brown alga, gymnosperm and angiosperm.

16. The deodorizing composition described in claim 15, wherein said plant derived deodorant component is at least one plant extract selected from a group comprising red alga, brown alia, gymnosperm and angiosperm.
